(19) United States
(12) Patent Application Publication
(10) Pub. No.: US 2018/0214668 A1
(43) Pub. Date: Aug. 2, 2018

(54) ANTI-AIR EMBOLISM SYSTEMS AND METHODS OF USING SAME

(71) Applicant: THE CLEVELAND CLINIC FOUNDATION, Cleveland, OH (US)

(72) Inventor: Mina K. Chung, Shaker Heights, OH (US)

(21) Appl. No.: 15/883,194

(22) Filed: Jan. 30, 2018

Related U.S. Application Data
(60) Provisional application No. 62/454,017, filed on Feb. 2, 2017.

(51) Int. Cl. A61M 25/01; A61B 18/14

(52) U.S. Cl. CPC A61M 25/01; A61B 18/1492; A61B 2018/00577; A61M 2205/18; A61M 2205/3331

(57) ABSTRACT

Systems and methods to reduce or minimize air embolisms from catheterization and other interventional procedures. Systems and methods are provided that include a chamber that can be filled with saline and hemostatically clipped to or incorporated with an open proximal end of a sheath for under saline catheter insertion, exchange, flushing or other steps that involve inserting a catheter into a patient's body.

### RELATED APPLICATIONS

The present application claims priority to U.S. Provisional Application No. 62/454,017, filed on Feb. 2, 2017, which is incorporated by reference herein.

### TECHNICAL FIELD

The present disclosure relates to systems and methods to reduce or minimize air embolisms from catheterization and other interventional procedures.

### BACKGROUND

Atrial fibrillation (AF) is one of the most common arrhythmias encountered in clinical practice and is characterized by uncoordinated atrial contraction. AF is associated with an increased risk of thrombo-embolic stroke as well as mortality relative to the general population. The onset of AF is believed to be due to triggers that initiate arrhythmia and a substrate that maintains it. Triggers of AF are thought to be localized to ectopic foci in the sleeves of the pulmonary vein ostia. As such, isolation of the pulmonary veins via catheter ablation has emerged as a routine treatment strategy to ameliorate the symptoms of AF. Although the procedure is effective, it is associated with certain complications. Major complications include stroke, transient ischemic attacks (TIAs) and ischemic brain lesions.

Several studies have identified silent central thrombo-embolism in AF ablation patients by magnetic resonance imaging (MRI). As these lesions are clinically silent, neurological examination does not identify clinical signs in these patients, although some lesions may result in stroke. Clinically silent micro-embolisms have been detected in patients undergoing PV ablation for AF, using diffusion weighted MRI one day after the ablation procedure. New embolic lesions have also been identified by MRI one day after ablation in patients undergoing left atrial radiofrequency (RF) ablation for AF. New silent cerebral ischemic lesions have been found in patients who underwent pulmonary vein isolation (PVI) with irrigated RF ablation, multielectrode catheter RF ablation, and cryoballoon ablation.

Until recently, little attention has been paid to the neurocognitive function of patients after AF ablation. However, it has been shown that some AF ablation patients have reduced verbal memory three months after ablation.

Micro-embolisms have occurred in patient despite the performance of AF ablation at high levels of anticoagulation. Air embolization may be an important contributor. When catheters are exchanged through a sheath or even flushed during or after these procedures, there is the possibility of introducing air emboli, as the hemostatic membranes in catheter systems are not air tight. Catheter exchanges can be attempted underwater, but it is not always feasible to obtain a saline container that allows the sheath to be underwater. Further, catheter exchange underwater is not feasible when there is only a short length of sheath extending from the groin, for example. Currently, air embolization is minimized with uninterrupted fluid through a catheter lumen but this does not solve the problem of embolization through the membranes of sheaths that are used during many interventional procedures such as left heart or arterial catheterization procedures. Accordingly, there is a need for an anti-air embolization system that can be used during catheterization procedures.

### SUMMARY OF THE DISCLOSURE

Aspects of the present disclosure are directed to anti-air embolism systems and methods of using the same.

In an embodiment, an anti-air embolism system comprises a sheath comprising a tubular body having a proximal portion and a distal portion. The system further comprises a chamber hemostatically attached to the proximal portion of the sheath. The chamber is sized and configured to accept a catheter. The system also includes a fluid source in communication with the chamber. Fluid is introduced to the chamber from the fluid source and is present in the chamber when a catheter is disposed therein. The system further includes an evacuation source in communication with the chamber configured to withdraw fluid or air from the chamber.

In another embodiment, a method of reducing air embolism during a catheterization procedure comprises inserting a sheath into a bodily lumen of a patient. The sheath has a proximal portion and a distal portion. The method further comprises hemostatically attaching a chamber to the proximal portion of the sheath. The chamber is sized and configured to accept a catheter. The method further includes providing fluid to the chamber and inserting a first catheter into the chamber and through the lumen of the sheath. The first catheter can be exchanged with a second catheter or flushed while fluid is present in the chamber.

### BRIEF DESCRIPTION OF THE DRAWINGS

FIG. 1 is a schematic illustration of an embodiment of an anti-air embolism system of the present disclosure.

FIG. 2 is a block diagram of an anti-air embolism system including a closed-loop pressure feedback system.

FIG. 3 is a block diagram of another embodiment of an anti-air embolism system including a closed-loop pressure feedback system.

FIG. 4 is a flow chart depicting steps of an embodiment of a method of the present disclosure.

### DETAILED DESCRIPTION

Aspects of the present disclosure are directed to reducing air microemboli during catherization and other interventional procedures. As used herein with respect to a described element, the terms “a,” “an,” and “the” include at least one or more of the described element unless including combinations thereof unless otherwise indicated. Further, the term “or” refers to “and/or” and combinations thereof unless otherwise indicated. In addition, it will be understood that when an element is referred to as being “on,” “attached” to, “connected” to, “coupled” with, “contacting,” or “in communication with,” another element, it can be directly on, attached to, connected to, coupled with, contacting or in communication with the other element or intervening elements may also be present. In contrast, when an element is referred to as being, for example, “directly on,” “directly attached” to, “directly connected” to, “directly coupled” with, “directly contacting,” or in “direct communication with” another element, there are no intervening elements present. It will also be appreciated by those of skill in the art that references to an element that is disposed “adjacent” another element may have portions that overlap or underlie the adjacent element. Further, as used herein, the term “patient” refers to a mammal, such as a human being.

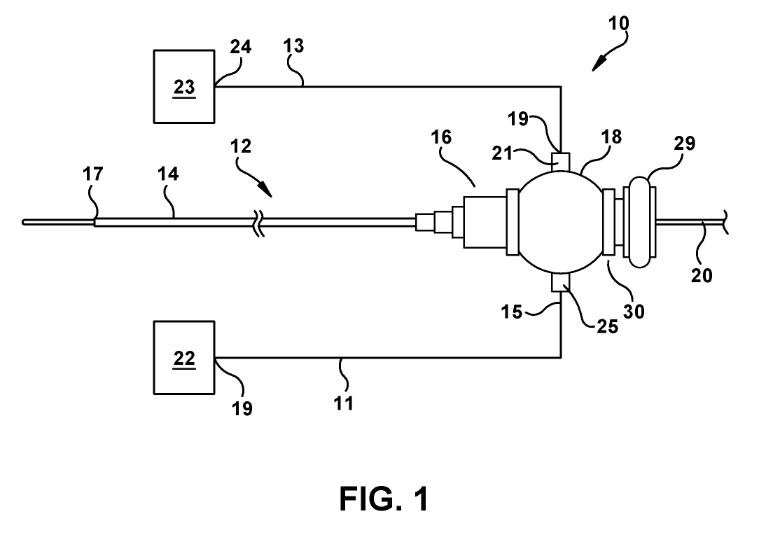

According to aspects of the present disclosure, anti-air embolism systems and methods include a chamber that can be filled with saline and hemostatically clipped to or incorporated with an open proximal end of a sheath for under saline catheter insertion, exchange, flushing or other steps that involve inserting a catheter into a patient's body. Referring to FIG. 1, in an embodiment, an anti-air embolism system 10 comprises a sheath 12 comprising a tubular body 14 having a proximal portion 16, a distal portion 17 and a lumen extending therebetween. System 10 further comprises a chamber 18 hemostatically attached to proximal portion 16 of sheath 12. Chamber 18 is sized and configured to accept a catheter 20. Chamber 18 can be fabricated from any suitable biocompatible material such as various plastics, polyurethane, or poly(vinyl) chloride. System 10 further includes a fluid source 22 that provides fluid to chamber 18. System 10 also includes an evacuation source 23 in communication with chamber 18 that is configured to withdraw fluid or air bubbles from chamber 18. System 10 can further include a leak proof seal or membrane 29 that can be attached to the distal end 30 of chamber 18 and through which catheter 20 can be inserted. Valve 29 can be an O-ring seal or a cross seal, for example, that hemostatically prevents or reduces blood or other bodily fluids from entering into chamber 18.

Fluid source 22 can be in communication with chamber 18 via an inflow line 11 that has one end 15 in fluid communication with an inlet port 25 of chamber 18 and another end 19 in fluid communication with fluid source 22. Evacuation source 23 can be in communication with chamber 18 via an outflow line 13 that has one end 19 in fluid communication with an output port 21 of chamber 18 and another end 24 in fluid communication with evacuation source 23. The fluid source and the evacuation source can be the same or different components. For example, the fluid source can be a pressurized fluid container that pressure infuses the chamber via the inflow line. The evacuation source can be a vacuum or suction that withdraws fluid and/or air from the chamber via the outflow line. Alternatively, the fluid source can be a syringe or similar device used to manually deliver fluid to the chamber and also to manually withdraw fluid from the chamber thereby acting as an evacuation source as well.

In use, fluid can be introduced into chamber 18 via fluid source 22. As stated above, the fluid can be pressure infused via input line 11 into chamber 18 or can be injected manually via a syringe into chamber 18. The fluid can be saline or any other sterile solution. Catheter 20 can be inserted into fluid-filled chamber 18 (optionally via valve 29) into the lumen of sheath 12 and through distal portion 17 of sheath 12. Because the insertion of catheter 20 into sheath 12 occurs in fluid (contained within chamber 18), the risk of air entering catheter 20, and therefore the risk of air embolism, is minimized. The necessary catheterization procedure then can be performed. Alternatively or additionally, if catheter 20 needs to be exchanged, it can be exchanged with another catheter in fluid-filled chamber 18. Similarly, if it is desired to flush catheter 20 with saline or another sterile solution, catheter 20 can be flushed in chamber 18 when fluid is disposed in chamber 18. The fluid and any air bubbles that may be present therein can be withdrawn from the chamber via evacuation source 23, such as a suction or vacuum source, or manually via a syringe. Fluid could also be pushed out of chamber 18, via a syringe or similar device inserted through valve 29. The fluid could be then be evacuated from chamber 18.

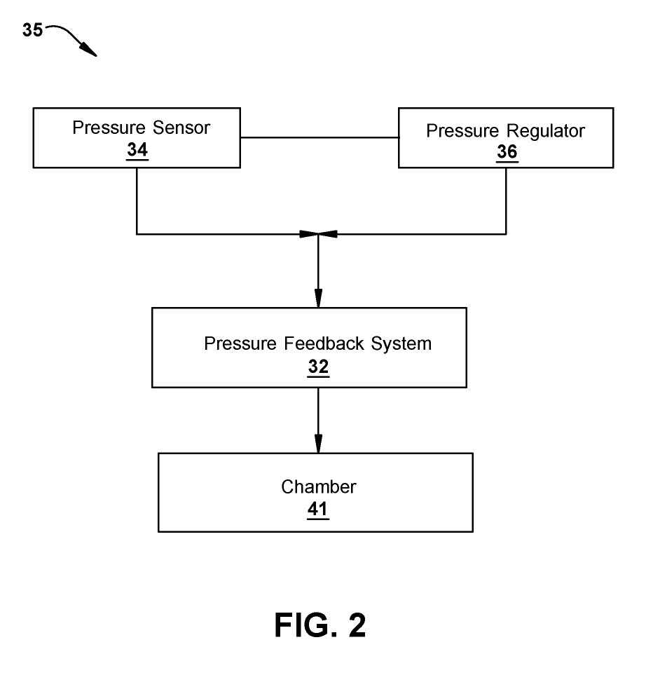

Referring to FIG. 2, in certain aspects, an anti-air embolism system 35 includes a closed-loop pressure feedback system 32 in communication with chamber 41 to regulate the fluid volume inside chamber 41. Pressure feedback system 32 can include pressure sensor 34 and pressure regulator 36. Pressure sensor 34 can sense the fluid volume inside chamber 41 and generate a sensor signal based on the sensed fluid volume. Based on the sensor signal, pressure regulator 36 can increase or decrease the fluid volume inside chamber 41 to control pressure head, dynamic pressure or both to ensure a proper differential pressure between the interior and exterior of the catheter. In other words, pressure regulator 36 can increase or decrease the fluid volume inside the chamber such that the interior catheter pressure is higher than the pressure at the valve of the anti-air embolism system. Such a system 32 can ensure that fluid flow is directed from the interior of the catheter towards the exterior of the catheter.

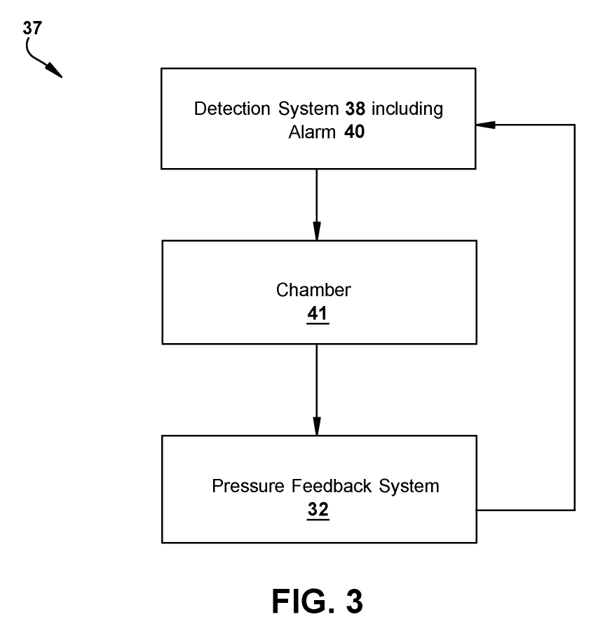

Referring to FIG. 3, in another embodiment, system 37 can also include a detection system 38 including an alarm 40 that alerts the user when the fluid volume inside chamber 41 is too high or too low. For example, alarm 40 can alert the user when there is a positive pressure differential or a negative pressure differential such that air bubbles may seep into the catheter. Such an alarm can be incorporated in a handle connected to chamber 41, for example. Pressure feedback system 32 can be in communication with chamber 41 by being integrated in chamber 41 or being a separate component outside of but connected to chamber 41, for example. Although FIGS. 2 and 3 depict a closed-loop pressure feedback system, an open loop feedback system can also be employed such that a user manually adjusts the fluid volume inside chamber 41 once the user is notified by detection system 38 that the fluid volume is incorrect.

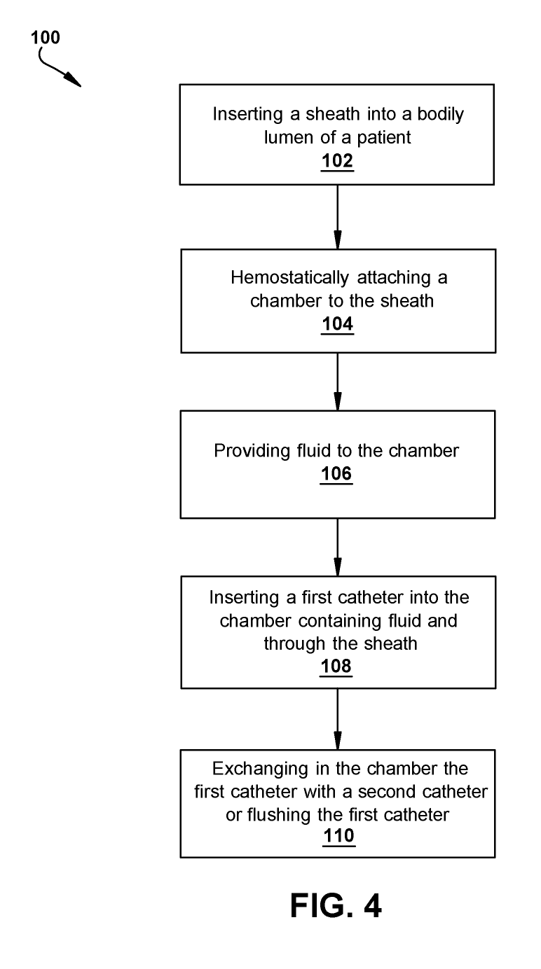

Aspects of the present disclosure provide methods of using an anti-air embolism chamber that can be incorporated into a sheath or clipped to a pre-existing sheath and therefore serve as an accessory to existing sheaths, such as trans-septal sheaths. Referring to FIG. 4, in an embodiment, a method 100 of reducing air embolism during a catheterization procedure comprises inserting a sheath into a bodily lumen of a patient 102. The sheath has a proximal portion, a distal portion and a lumen extending therebetween. The method further includes hemostatically attaching a chamber to the proximal portion of the sheath 104. The chamber is sized and configured to accept a catheter. Fluid can be introduced into the chamber 106. A first catheter can be inserted into the fluid-filled chamber and through the lumen of the sheath 108. While fluid is disposed in the chamber, the first catheter can be exchanged with a second catheter or the first catheter can be flushed with a solution to cleanse the catheter 110. The fluid can be removed from the chamber during or after catheter insertion, catheter exchange or catheter flushing. Air bubbles present in the fluid can be removed during catheter insertion, catheter exchange or catheter flushing.

Methods and systems as described herein can be used to reduce air microemboli during various catheterization and interventional procedures. Such procedures include, for example, vascular procedures including left sided cardiac catheterization, right sided cardiac catheterization, peripheral vascular intervention, and percutaneous coronary intervention. Such procedures also include arterial catheterization and venous catheterization. The catheterization procedures can be used for various electrophysiological ablation and other interventional procedures. For example, the catheterization procedures can be used for cardiac ablation, such as left-sided cardiac ablation. The cardiac ablation can be used to treat various conditions such as AF, atrial tachycardia, and ventricular tachycardia (VT), for example. Further, the methods and systems as described herein can be used in other procedures where it is undesirable to introduce air into a catheter or tube. Such applications include, for example, hemodialysis or perfusion systems where it desirable to use a fluid barrier.

Each of the disclosed aspects and embodiments of the present disclosure may be considered individually or in combination with other aspects, embodiments, and variations of the disclosure. Further, while certain features of embodiments of the present disclosure may be shown in only certain figures, such features can be incorporated into other embodiments shown in other figures while remaining within the scope of the present disclosure. In addition, unless otherwise specified, none of the steps of the methods of the present disclosure are confined to any particular order of performance. Furthermore, all references cited herein are incorporated by reference in their entirety.

What is claimed is:

1. An anti-air embolism system comprising:
a sheath comprising a tubular body having a proximal portion and a distal portion;
a chamber hemostatically attached to the proximal portion of the sheath, the chamber being sized and configured to accept a catheter;
a fluid source in communication with the chamber, wherein fluid from the fluid source is present in the chamber when a catheter is disposed therein; and
an evacuation source in communication with the chamber configured to withdraw fluid or air from the chamber.

2. The system of claim 1, further comprising:
an inflow line having one end in fluid communication with an inlet port of the chamber and another end in fluid communication with the fluid source; and
an outflow line having one end in fluid communication with an outlet port of the chamber and another end in fluid communication with the evacuation source.

3. The system of claim 2, wherein the fluid source is a pressurized fluid container.

4. The system of claim 2, wherein the evacuation source is a vacuum or suction source.

5. The system of claim 1, wherein the fluid source is a syringe.

6. The system of claim 1, wherein the evacuation source is a syringe.

7. The system of claim 1, further comprising a leak-proof membrane attached to a distal end of the chamber.

8. The system of claim 1, further comprising a closed-loop pressure feedback system in communication with the chamber, the pressure feedback system comprising:
a pressure sensor that senses the fluid volume inside the chamber and generates a sensor signal based on the sensed fluid volume; and
a pressure regulator that adjusts the fluid volume inside the chamber based on the sensor signal such that fluid flow is directed from the interior of the catheter towards the exterior of the catheter.

9. The system of claim 8, further comprising a detection system including an alarm programmed to alert a user when there is a positive or negative pressure differential in the chamber.

10. A method of reducing air embolism during a catheterization procedure comprising:
inserting a sheath into a bodily lumen of a patient, the sheath having a proximal portion and a distal portion;
hemostatically attaching a chamber to the proximal portion of the sheath, the chamber being sized and configured to accept a catheter;
providing fluid to the chamber; and
inserting a first catheter into the chamber containing fluid and through the lumen of the sheath.

11. The method of claim 10, wherein the bodily lumen is a blood vessel.

12. The method of claim 10, further comprising exchanging in the chamber the first catheter with a second catheter or flushing the first catheter while the fluid is present in the chamber.

13. The method of claim 10, further comprising withdrawing the fluid from the chamber during or after inserting the first catheter into the chamber and through the lumen of the sheath.

14. The method of 10, further comprising removing air bubbles from the fluid in the chamber during insertion of the first catheter through the lumen of the sheath.

15. The method of claim 12, further comprising withdrawing the fluid from the chamber during or after exchanging the first catheter with the second catheter or flushing the first catheter.

16. The method of claim 12, further comprising removing air bubbles from the fluid in the chamber during exchanging the first catheter with the second catheter or flushing the first catheter.

17. The method of claim 10, wherein the sheath is a trans-septal sheath.

18. The method of claim 10, wherein the catheterization procedure is a cardiac ablation procedure.

19. The method of claim 10, wherein the catheterization procedure is a left sided or right sided cardiac catheterization procedure.

20. The method of claim 10, wherein the catheterization procedure is a hemodialysis or perfusion procedure.
